(19) United States
(12) Patent Application Publication
(10) Pub. No.: US 2003/0032886 A1
(43) Pub. Date: Feb. 13, 2003

(54) System for determining coronary flow reserve (CFR) value for a stenosed blood vessel, CFR processor therefor, and method therefor

(76) Inventors: Elhanan Dgany, Kfar Saba (IL); Orly Dgany, Kfar Saba (IL); Simon Henri Noskowicz, Kfar Saba (IL); Evgeny Shalman, Tel Aviv (IL); Chen Barak, Shoam (IL)

Correspondence Address: Harold L. Novick, NATH & ASSOCIATES PLLC, 6th Floor, 1030 15th Street, N.W., Washington, DC, 20005, US

(21) Appl. No.: 10/108,999

(22) Filed: Mar. 29, 2002

Related U.S. Application Data
(63) Continuation-in-part of application No. 09/264,782, filed on Mar. 9, 1999, now abandoned.
(63) Continuation-in-part of application No. 09/344,505, filed on Jun. 25, 1999, now Pat. No. 6,471,656.
(63) Continuation-in-part of application No. 09/666,841, filed on Sep. 21, 2000, now abandoned.
(60) Provisional application No. 60/123,499, filed on Mar. 9, 1999.
(60) Provisional application No. 60/140,799, filed on Jun. 25, 1999.
(60) Provisional application No. 60/155,095, filed on Sep. 22, 1999.

(51) Int. Cl. A61B 5/02

(52) U.S. Cl. 600/487

(57) ABSTRACT

The present invention is directed toward determining a Coronary Flow Reserve (CFR) value for a stenosed blood vessel by way of pressure measurements acquisitioned proximal a stenosis in a stenosed blood vessel and distal thereto both at rest and at hyperemia in accordance with the relationship CFR α (ΔP_{hyper}, ΔPᵣₑₛₜ) where ΔP_{hyper} and ΔPᵣₑₛₜ are the pressure gradients across the stenosis at rest and at hyperemia, respectively.

### FIELD OF THE INVENTION

The invention relates to determining the values for intravascular hemodynamic parameters in general, and the coronary flow reserve value for a stenosed blood vessel in particular.

### BACKGROUND OF THE INVENTION

Vascular diseases are often manifested by reduced blood flow due to atherosclerotic occlusion of vessels. Two hemodynamic parameters coronary flow reserve and fractional flow reserve are often used by clinicians to determine the severity of a stenosed vessel for prognostic purposes.

Coronary Flow Reserve (CFR) is defined as the ratio of mean hyperemic flow to mean flow at rest, namely,

$$CFR = \frac{{\int }_{h e a t b e a t} {Q}_{h y p e r} ⅆ t}{{\int }_{h e a t b e a t} {Q}_{r e s t} ⅆ t}$$

The flow measurements are typically acquisitioned using a Doppler guide wire, for example, commercially available from Cardiometrics Inc., California, USA. Clinical studies have demonstrated that in most cases stenosed vessels yielding CFR<2 must be treated whilst angioplasty may be avoided for stenosed vessels yielding CFR>2.

Fractional Flow Reserve (FFR) is defined as the ratio FFR=PvB/PvA where PvB and PvA are the mean pressures over a heartbeat during hyperemia respectively distal to a stenosis, and proximal thereto. The pressure measurements are typically acquisitioned using a pressure guide wire, for example, the Pressure Wire™ commercially available from Radi Medical System, Uppsala, Sweden. Clinical studies have shown that angioplasty may be avoided in most cases yielding FFR>0.75.

Clinical studies (Di Mario et al., 1996) show that CFR and FFR lead to the same medical conclusions regarding stenosis significance for around 75% of patient cases implying that determination of both parameters will provide improved information for prognostic purposes. Up to the present time, simultaneous use of both flow and pressure guide wires has been confined to research, and determination of both parameters has required two consecutive procedures with the inherent drawbacks of cost, time and patient safety.

### SUMMARY OF THE INVENTION

The present invention is based on the realization that a Coronary Flow Reserve (CFR) value for a stenosed blood vessel can be determined from pressure measurements acquisitioned proximal to a stenosis and distal thereto instead of flow measurements, thereby enabling simultaneous determination of both CFR and FFR values in the same intravascular procedure. Moreover, it is envisaged that a CFR value in accordance with the present invention may be more accurate than hitherto possible since pressure guide wires are generally considered more reliable than flow guide wires.

In an article entitled “In Vitro Study of Pressure-Velocity Relation Across Stenostic Orifices”, Wong et al., American Journal of Cardiology, v.56, pp.465-469), it was shown that the pressure gradient—blood flow relationship for a short stenosis is independent of orifice size over a wide pressure range. More particularly, the relationship was found to be quadratic and crossing zero, namely, Δp=const Q² where the const is determined by stenosis diameter only and requires an additional measurement. In the case of arbitrary long stenoses, the pressure gradient across a stenosis can be modeled as follows: Δp=K₁Q+K₂Q²+K₃dQ/dt, as illustrated and described in an article entitled “Flow characteristics in model of arterial stenosis”—II. Unsteady flow, Young et al., Journal of Biomechanics, 1973, vol. 6, pp.547-559. However, it has been found that the first and last members of this equation are relatively insignificant, thereby effectively reducing this equation also to Δp=const Q².

Based on the above, and as substantiated by empirical findings, the conventional CFR equation can be accurately approximated by the following relationship:

$$CFR = \sqrt{\frac{Δ {P}_{h y p e r}}{Δ {P}_{r e s t}}}$$

where ΔP_{hyper} is the pressure gradient across the stenosis at hyperemia and ΔPᵣₑₛₜ is the pressure gradient across the stenosis at rest. In accordance with the preferred embodiment of the present invention,

$$Δ {P}_{h y p e r} = {\int }_{h e a r t b e a t} {( {P ( t )}_{p r o x i m a l} - {P ( t )}_{d i s t a l} )}_{h y p e r} ⅆ t a n d$$

$$Δ {P}_{r e s t} = {\int }_{h e a r t b e a t} {( {P ( t )}_{p r o x i m a l} - {P ( t )}_{d i s t a l} )}_{r e s t} ⅆ t$$

Alternatively, ΔP_{hyper}=max(P(i)ₚᵣₒₓᵢₘₐₗ−P(i)_{distal})_{hyper} over a heartbeat whilst ΔPᵣₑₛₜ=max(P(i)ₚᵣₒₓᵢₘₐₗ−P(i)_{distal})ᵣₑₛₜ over a heartbeat where P(i) is a sample pressure measurement. The latter approach may be employed when the former approach does not yield sufficient high values for ΔP_{hyper} and ΔPᵣₑₛₜ.

### BRIEF DESCRIPTION OF THE DRAWINGS

In order to understand the invention and to see how it can be carried out in practice, preferred embodiments will now be described, by way of non-limiting examples only, with reference to the accompanying drawings in which:

FIG. 1 is a block diagram of a system including a CFR processor for determining the CFR value for a stenosed blood vessel;

FIG. 2 is a graph showing exemplary pressure waveforms of pressure measurements simultaneously acquisitioned by a fluid filled pressure transducer and a pressure guide wire both proximal to a stenosis within a stenosed blood vessel at rest;

FIG. 3 is a graph showing exemplary pressure waveforms of pressure measurements simultaneously acquisitioned by a fluid filled pressure transducer proximal to a stenosis within a stenosed blood and a pressure guide wire distal thereto at rest;

FIG. 4 is a graph showing exemplary pressure waveforms of pressure measurements simultaneously acquisitioned by a fluid filled pressure transducer proximal to a stenosis within a stenosed blood vessel and a pressure guide wire distal thereto at hyperemia;

FIG. 5 is a flow diagram showing the steps executed by the CFR processor of FIG. 1 for determining the CFR value for a stenosed blood vessel;

FIG. 6 is a graph showing pressure pulses acquisitioned by the pressure guide wire of the system of FIG. 1 before synchronization;

FIG. 7 is a graph showing the pressure pulses of FIG. 6 after synchronization;

FIG. 8 is a graph showing a mean proximal pressure pulse at rest, a mean distal pressure pulse at rest, and a distal pressure pulse hyperemia; and

FIG. 9 is a graph showing the selection of the third largest CFR value as the actual CFR value for a stenosed blood vessel.

### DETAILED DESCRIPTION OF THE DRAWINGS

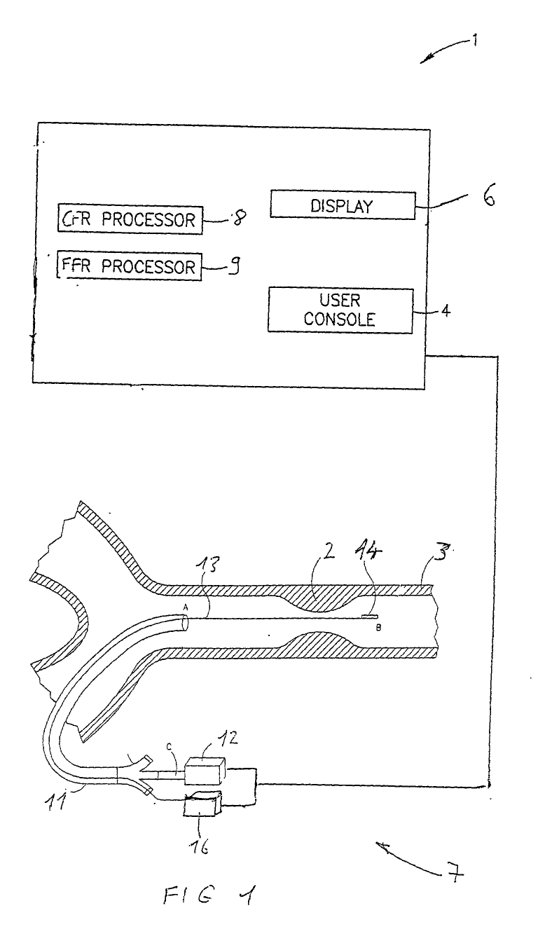

FIG. 1 shows a system 1 for diagnosing severity of a stenosis 2 in a stenosed blood vessel 3 to determine the necessity of medical treatment of the stenosis, and the type of such treatment. The system 1 is under the control of a user console 4 including a display 6, and includes intravascular pressure measurement apparatus 7 for acquisitioning pressure waveforms both proximal to the stenosis and distal thereto. The system 1 includes a CFR processor 8 programmed for calculating the CFR value for the stenosed blood vessel 2, and a FFR processor 9 for programmed for determining the FFR value for the stenosed blood vessel in accordance with the above mentioned relationship FFR=PvB/PvA. In a preferred embodiment of the present invention, the user console 4, the display 6, the CFR processor 8 and the FFR processor 9 are embodied as a general purpose digital computer.

The intravascular pressure measurement apparatus 7 includes a guiding catheter 11 connected to a fluid filled pressure transducer 12. The fluid filled pressure transducer 12 acquisitions pressure measurements outside of patient's body at position C for use as a reference signal (see FIG. 1). An exemplary guiding catheter 11 is the Ascent JL4 catheter commercially available from Medtronic, USA whilst an exemplary fluid filled pressure transducer 12 is commercially available from Biometrix, Jerusalem, Israel. The intravascular pressure measurement apparatus 7 also includes a pressure guide wire 13 with a pressure transducer 14 at its tip connected to a signal processing device 16 for acquisitioning pressure measurements at positions A and B respectively proximal and distal to the stenosis 2 (see FIG. 1). An exemplary pressure guide wire 13 is the PressureWire™ pressure guide wire whilst an exemplary signal conditioning device 16 is a TCB-500 model commercially available from Millar Instruments, USA.

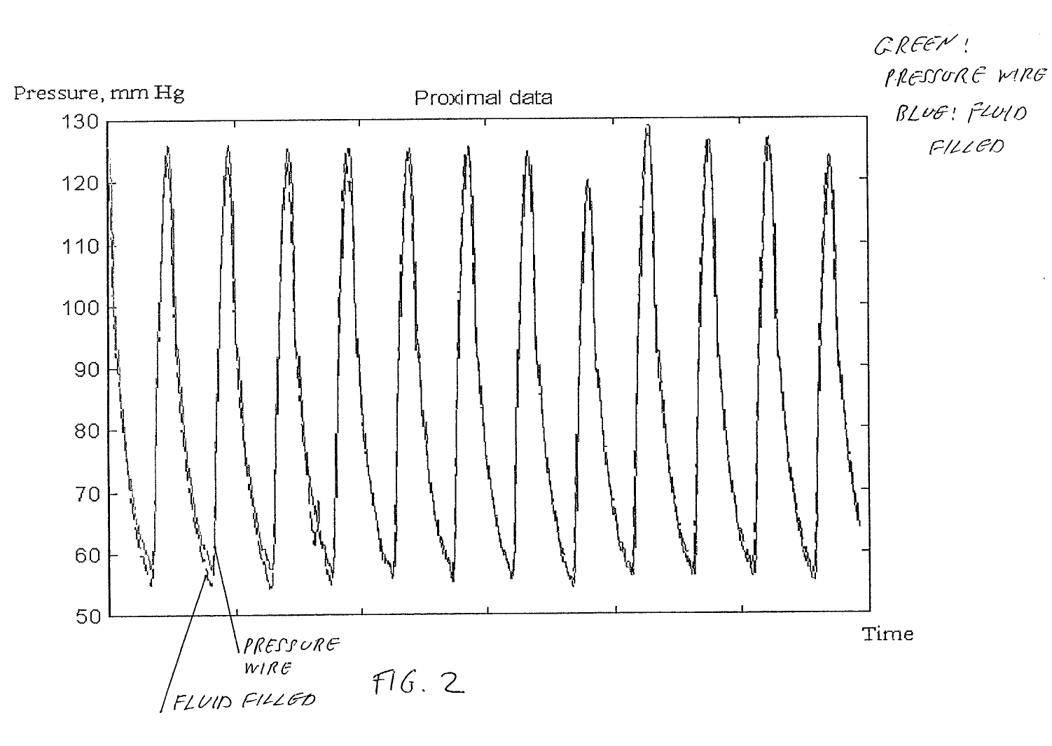

The use of the system 1 for determining the CFR value for the stenosed blood vessel 3 under investigation is now described with reference to FIGS. 2-4:

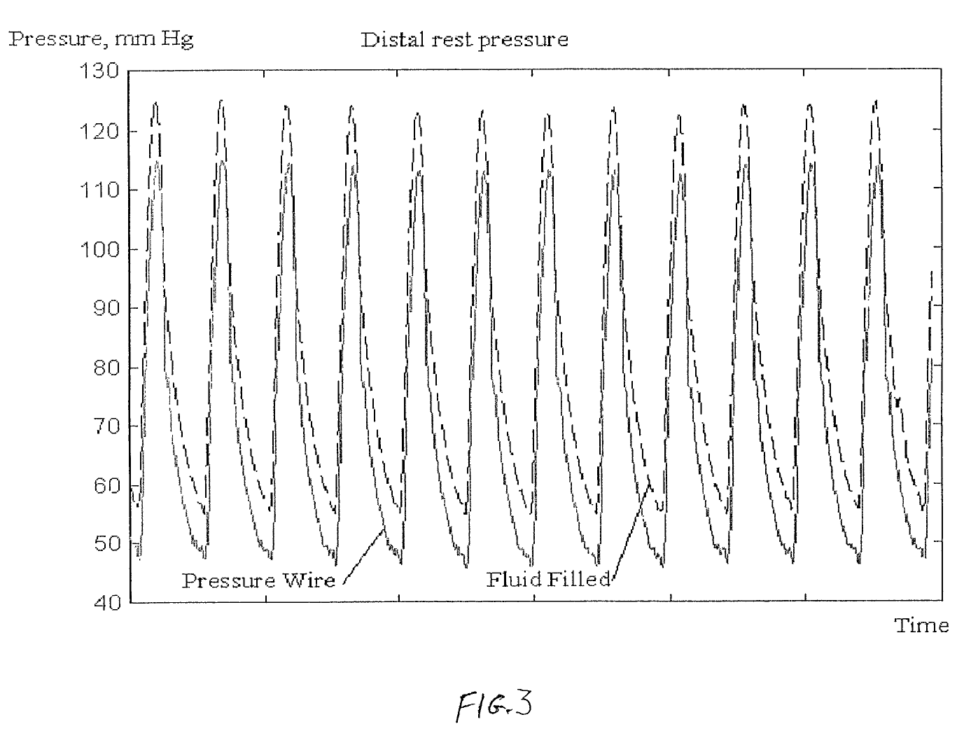

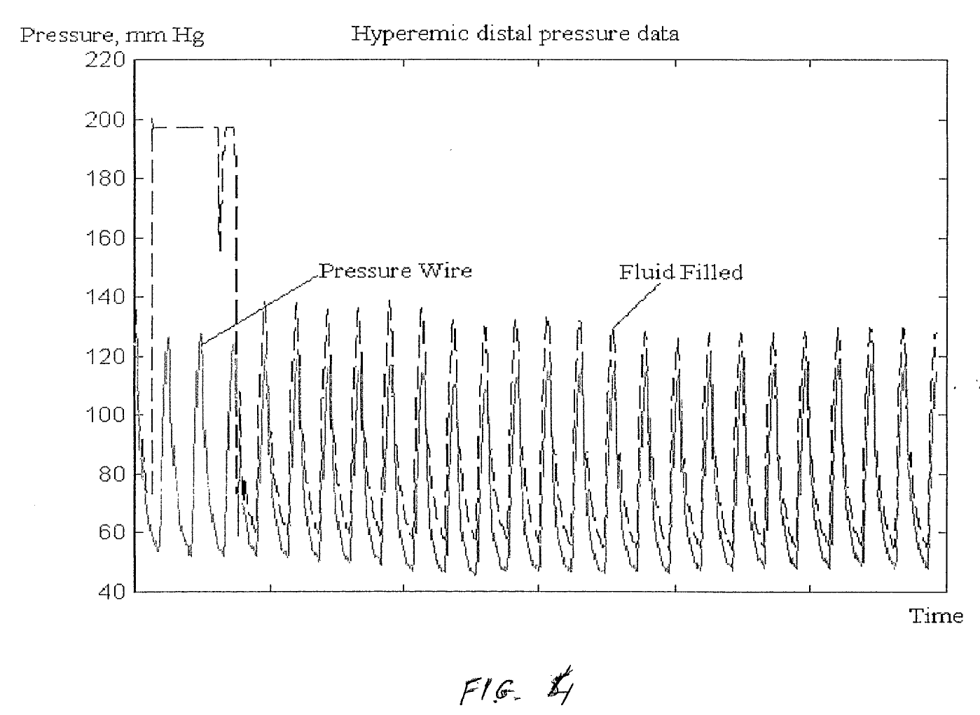

The guiding catheter 11 is introduced into the stenosed blood vessel 3 to position A at rest. The pressure guide wire 13 is introduced into the guiding catheter 11 such that its pressure transducer 14 lies approximately flush with the catheter's tip. Pressure measurements are simultaneously acquisitioned by both the pressure transducer 12 and the pressure guide wire 13 (see FIG. 2). As shown, the two pressure measurements are almost entirely superimposed one upon the other. The pressure guide wire 13 is further introduced into the guiding catheter 11 to position B such that its pressure transducer 14 extends distally beyond the stenosis 2 under investigation whereat pressure measurements are again simultaneously acquisitioned by both the pressure transducer 12, and the pressure guide wire 13 at position B (see FIG. 3). After acquisitioning the pressure measurements distal to the stenosis 2 at rest, hyperemia is induced by administration of a suitable medicament, for example adehnosin. Pressure measurements are again simultaneously acquisitioned by both the pressure transducer 12, and the pressure guide wire 13 at position B (see FIG. 4).

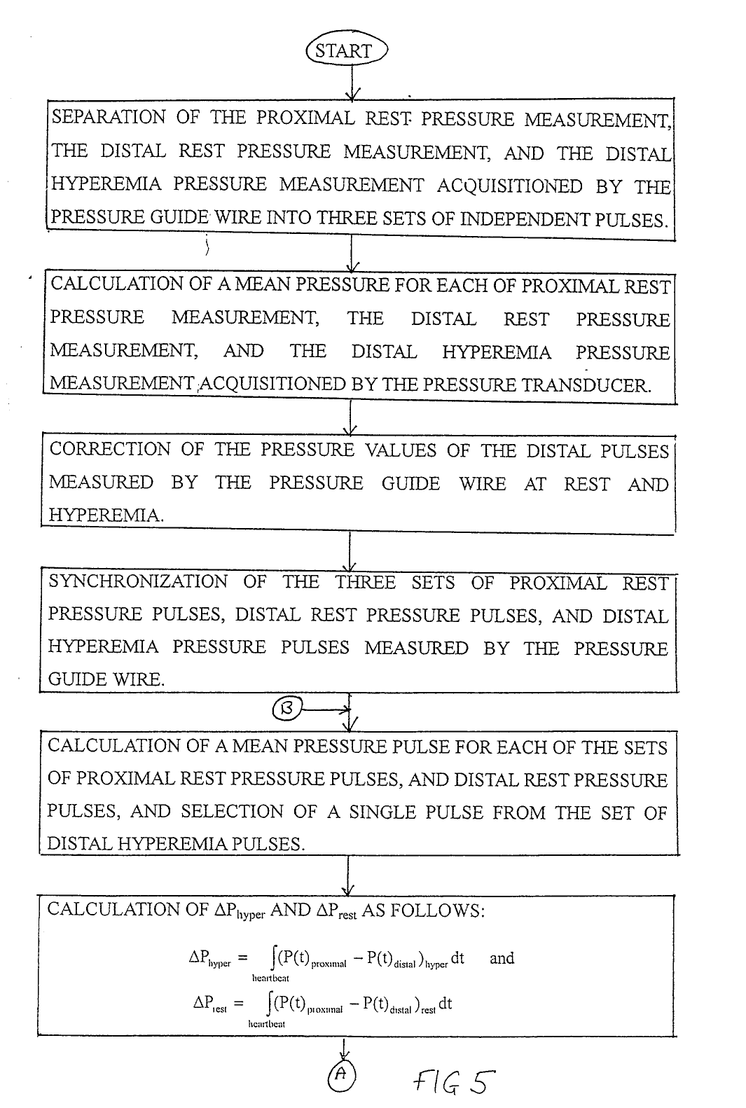

Upon acquisitioning the pressure measurements, the CFR processor 8 executes the following steps to determine the CFR value for the stenosed blood vessel 3 (see FIGS. 5-9):

Step 1: Separation of the proximal rest pressure measurement, the distal rest pressure measurement, and the distal hyperemia pressure measurement acquisitioned by the pressure guide wire into three sets of independent pulses each starting and ending at a local minimum.

Step 2: Calculation of a mean pressure for each of proximal rest pressure measurement, the distal rest pressure measurement, and the distal hyperemia pressure measurement acquisitioned by the pressure transducer denoted mean P_{C1}, mean P_{C2}, and mean P_{C3} for determining amplification factors K_{2,1}=mean P_{C2}/mean PC₁ and K_{3,1}=mean P_{C3}/mean P_{C1}.

Step 3: Correction of the pressure values of the distal pulses measured by the pressure guide wire at rest and hyperemia using the factors K_{2,1} and K_{3,1}, respectively, to compensate for the fact various factors, for example, breathing, patient movement, and the like, may influence the measurements since they were not acquisitioned simultaneously.

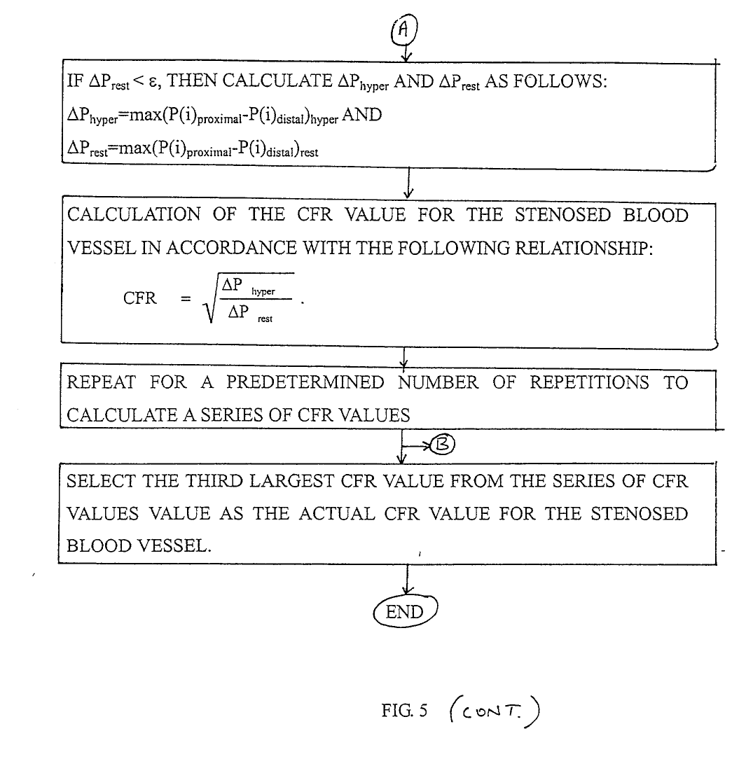

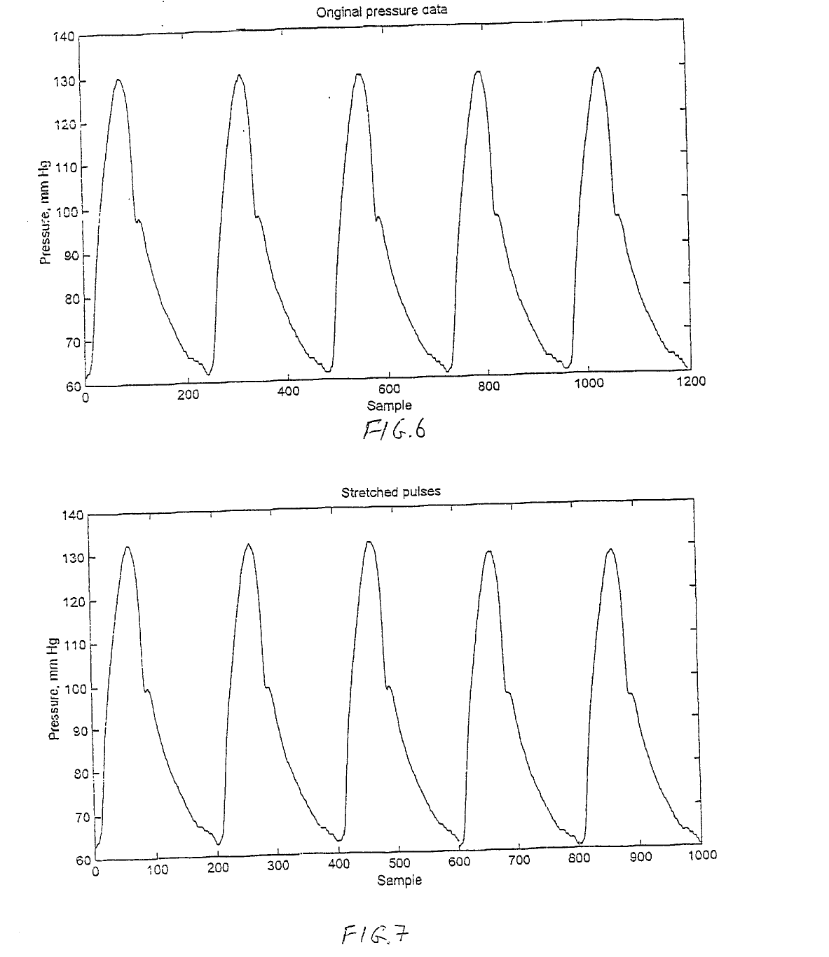

Step 4: Synchronization of the three sets of proximal rest pressure pulses, distal rest pressure pulses, and distal hyperemia pressure pulses measured by the pressure guide wire by normalizing their width such that each pulse of each set of independent pulses has the same period between local minimum values i.e. the width of each pulse is stretched or compacted as necessary. FIGS. 6 and 7 illustrate exemplary pressure pulses before and after synchronization, respectively.

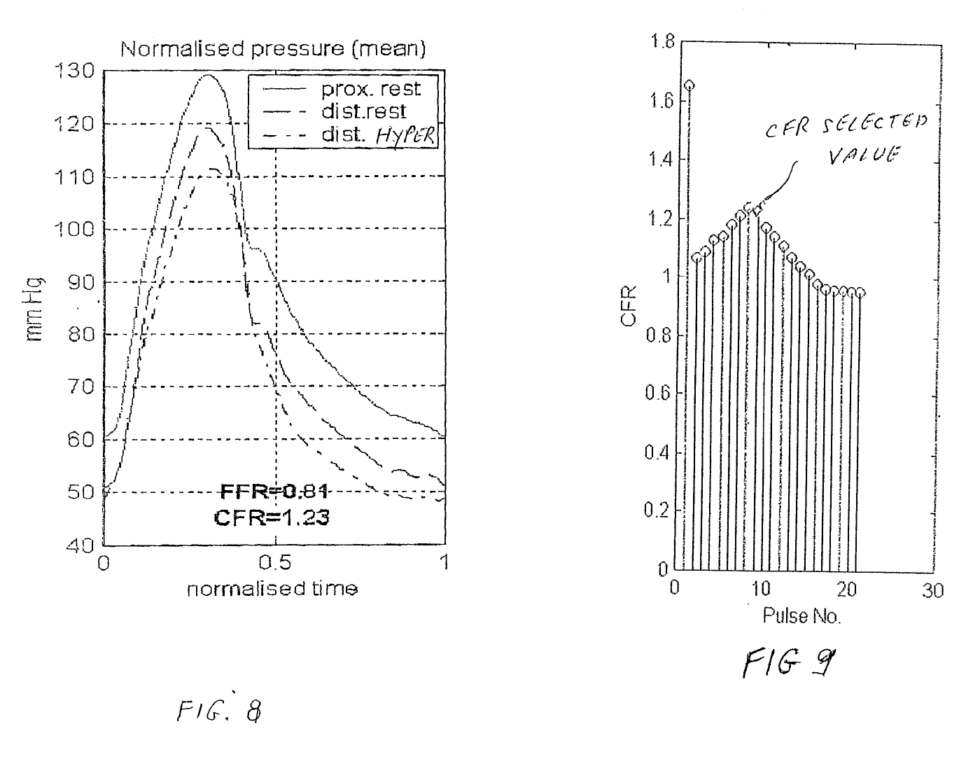

Step 5: Calculation of a mean pressure pulse for each of the sets of proximal rest pressure pulses, and distal rest pressure pulses, and selection of a single pulse from the set of distal hyperemia pulses (see FIG. 8).

Step 6: Calculation of ΔP_{hyper} and ΔPᵣₑₛₜ as follows:

$$Δ {P}_{h y p e r} = {\int }_{h e a r t b e a t} {( {P ( t )}_{p r o x i m a l} - {P ( t )}_{d i s t a l} )}_{h y p e r} ⅆ t a n d$$

$$Δ {P}_{r e s t} = {\int }_{h e a r t b e a t} {( {P ( t )}_{p r o x i m a l} - {P ( t )}_{d i s t a l} )}_{r e s t} ⅆ t$$

where it is assumed that the proximal hyperemic pressure pulse which was not measured is equal to the proximal rest pressure pulse.

Step 7: If ΔPᵣₑₛₜ<ε, where ε is a predetermined cutoff value, say, 1 mm Hg, then calculate ΔP_{hyper} and ΔPᵣₑₛₜ as follows: ΔP_{hyper}=max(P(i)ₚᵣₒₓᵢₘₐₗ−P(i)_{distal})_{hyper} over a heartbeat where P(i) is a sample pressure measurement, and similarly ΔPᵣₑₛₜ=max(P(i)ₚᵣₒₓᵢₘₐₗ−P(i)_{distal})ᵣₑₛₜ over a heartbeat where P(i) is a sample pressure measurement.

Step 8: Calculation of the CFR value for the stenosed blood vessel in accordance with the following relationship:

$$CFR = \sqrt{\frac{Δ {P}_{h y p e r}}{Δ {P}_{r e s t}}}$$

where the values ΔP_{hyper} and ΔPᵣₑₛₜ are obtained from either Step 6 or Step 7.

Step 9: Repeat Steps 5 to 8 for a predetermined number of repetitions, say, 21 repetitions as shown in FIG. 9, each time using a different pulse from the set of distal hyperemia pressure pulses to calculate a series of CFR values for the stenosed blood vessel for selection of typically the third largest CFR value as the actual CFR value for the stenosed blood vessel.

While the invention has been described with respect to a limited number of embodiments, it will be appreciated that many variations, modifications, and other applications of the invention can be made within the scope of the appended claims. For example, rather than relying on the pressure measurement at point A at rest for determining ΔP_{hyper}, a pressure guide wire can be withdrawn to Point A. Alternatively, a pair of pressure guide wires can be introduced into the guiding catheter for acquisitioning pressure measurements simultaneously proximal to the stenosis and distal thereto. Along these lines, a pressure guide wire with a pair of spaced apart pressure transducers and can be employed for acquisitioning pressure measurements simultaneously proximal to a stenosis, and distal thereto.

1. A system for determining a Coronary Flow Reserve (CFR) value for a stenosed blood vessel, the system comprising:
(a) intravascular pressure measurement apparatus for acquisitioning pressure measurements proximal to the stenosis in the stenosed blood vessel and distal thereto both at rest and at hyperemia; and
(b) a Coronary Flow Reserve (CFR) processor for determining the CFR value in accordance with the relationship
$$CFR = \sqrt{\frac{Δ {P}_{h y p e r}}{Δ {P}_{r e s t}}}$$
where ΔP_{hyper} and ΔPᵣₑₛₜ are pressure gradients across the stenosis at hyperemia and at rest, respectively.

2. The system according to claim 1 wherein
$$Δ {P}_{h y p e r} = {\int }_{h e a r t b e a t} {( {P ( t )}_{p r o x i m a l} - {P ( t )}_{d i s t a l} )}_{h y p e r} ⅆ t a n d$$
$$Δ {P}_{r e s t} = {\int }_{h e a r t b e a t} {( {P ( t )}_{p r o x i m a l} - {P ( t )}_{d i s t a l} )}_{r e s t} ⅆ t$$

3. The system according to claim 1 wherein
ΔP_{hyper}=max(P(i)ₚᵣₒₓᵢₘₐₗ−P(i)_{distal})_{hyper} over a heartbeat,
ΔPᵣₑₛₜ=max(P(i)ₚᵣₒₓᵢₘₐₗ−P(i)_{distal})ᵣₑₛₜ over a heartbeat, and P(i) is a sample pressure measurement.

4. The system according to claim 1 wherein a pressure gradient ΔP is derived from a pair of simultaneously acquisitioned pressure measurements acquisitioned proximal to the stenosis and distal thereto.

5. The system according to claim 4 wherein a fluid filled pressure transducer acquisitions the pressure measurement proximal to the stenosis and a pressure guide wire acquisitions the pressure measurement distal to the stenosis.

6. The system according to claim 1 wherein a pressure gradient ΔP is derived from a pair of consecutively acquisitioned pressure measurements acquisitioned proximal to the stenosis and distal thereto, and said CFR processor employs a heartbeat related physiological signal for synchronizing the pressure measurement acquisitioned proximal to the stenosis with the pressure measurement acquisitioned distal to the stenosis.

7. The system according to claim 6 wherein a pressure guide wire acquisitions said pair of consecutively acquisitioned pressure measurements, and a fluid filled manometer acquisitions said heartbeat related physiological signal.

8. For use with intravascular pressure measurement apparatus capable of acquisitioning pressure measurements proximal to the stenosis in a stenosed blood vessel and distal thereto both at rest and at hyperemia, a Coronary Flow Reserve (CFR) processor for determining a CFR value for the stenosed blood vessel, the CFR processor capable of executing the following steps:
(a) determining a pressure gradient ΔPᵣₑₛₜ across the stenosis at rest;
(b) determining a pressure gradient ΔP_{hyper} across the stenosis at hyperemia; and
(c) determining the CFR value in accordance with the relationship:
$$CFR = \sqrt{\frac{Δ {P}_{h y p e r}}{Δ {P}_{r e s t}}} .$$

9. The CFR processor according to claim 8 wherein:
$$Δ {P}_{h y p e r} = {\int }_{h e a r t b e a t} {( {P ( t )}_{p r o x i m a l} - {P ( t )}_{d i s t a l} )}_{h y p e r} ⅆ t a n d$$
$$Δ {P}_{r e s t} = {\int }_{h e a r t b e a t} {( {P ( t )}_{p r o x i m a l} - {P ( t )}_{d i s t a l} )}_{r e s t} ⅆ t .$$

10. The CFR processor according to claim 8 wherein:
ΔP_{hyper}=max(P(i)ₚᵣₒₓᵢₘₐₗ−P(i)_{distal})_{hyper} over a heartbeat,
ΔPᵣₑₛₜ=max(P(i)ₚᵣₒₓᵢₘₐₗ−P(i)_{distal})ᵣₑₛₜ over a heartbeat, and P(i) is a sample pressure measurement.

11. The CFR processor according to claim 8 wherein a pressure gradient ΔP is derived from a pair of simultaneously acquisitioned pressure measurements acquisitioned proximal to the stenosis and distal thereto.

12. The CFR processor according to claim 11 wherein a pressure gradient ΔP is derived from a pair of consecutively acquisitioned pressure measurements acquisitioned proximal to the stenosis and distal thereto, and said CFR processor employs a heartbeat related physiological signal for synchronizing the pressure measurement acquisitioned proximal to the stenosis with the pressure measurement acquisitioned distal to the stenosis.

13. A method for determining a Coronary Flow Reserve (CFR) value for a stenosed blood vessel, the method comprising the steps of:
(a) acquisitioning pressure measurements proximal to the stenosis in the stenosed blood vessel and distal thereto both at rest and at hyperemia; and
(b) determining the CFR value in accordance with the relationship:
$$CFR = \sqrt{\frac{Δ {P}_{h y p e r}}{Δ {P}_{r e s t}}}$$
where ΔP_{hyper} and ΔPᵣₑₛₜ are pressure gradients across the stenosis at hyperemia and at rest, respectively.

14. The method according to claim 13 wherein:
$$Δ {P}_{h y p e r} = {\int }_{h e a r t b e a t} {( {P ( t )}_{p r o x i m a l} - {P ( t )}_{d i s t a l} )}_{h y p e r} ⅆ t a n d$$
$$Δ {P}_{r e s t} = {\int }_{h e a r t b e a t} {( {P ( t )}_{p r o x i m a l} - {P ( t )}_{d i s t a l} )}_{r e s t} ⅆ t$$

15. The method according to claim 13 wherein:
ΔP_{hyper}=max(P(i)ₚᵣₒₓᵢₘₐₗ−P(i)_{distal})_{hyper} over a heartbeat,
ΔPᵣₑₛₜ=max(P(i)ₚᵣₒₓᵢₘₐₗ=P(i)_{distal})ᵣₑₛₜ over a heartbeat, and P(i) is a sample pressure measurement.

16. The method according to claim 13 wherein a pressure gradient ΔP is derived from a pair of simultaneously acquisitioned pressure measurements acquisitioned proximal to the stenosis and distal thereto.

17. The method according to claim 16 wherein a fluid filled pressure transducer acquisitions the pressure measurement proximal to the stenosis and a pressure guide wire acquisitions the pressure measurement distal to the stenosis.

18. The method according to claim 13 wherein a pressure gradient ΔP is derived from a pair of consecutively acquisitioned pressure measurements acquisitioned proximal to the stenosis and distal thereto, and further comprising the step of employing a heartbeat related physiological signal for synchronizing the pressure measurement acquisitioned proximal to the stenosis with the pressure measurement acquisitioned distal to the stenosis.

19. The method according to claim 18 wherein a pressure guide wire acquisitions the pair of consecutively acquisitioned pressure measurements, and a fluid filled manometer acquisitions the heartbeat related physiological signal.
